(19) United States
(12) Patent Application Publication
(10) Pub. No.: US 2009/0088699 A1
(43) Pub. Date: Apr. 2, 2009

(54) Continuous Multi-Geometric Profile Catheter

(76) Inventors: Eric King, West Jordan, UT (US); Ronald Wortley, Salt Lake City, UT (US)

Correspondence Address: KENNETH E. HORTON;KIRTON & MCCONKLE, 60 EAST SOUTH TEMPLE, SUITE 1800, SALTLAKE CITY, UT, 84111, US

(21) Appl. No.: 12/234,392

(22) Filed: Sep. 19, 2008

Related U.S. Application Data
(60) Provisional application No. 60/976,290, filed on Sep. 28, 2007.

(51) Int. Cl. A61M 25/02; A61M 25/00; A61M 25/18

(52) U.S. Cl. 604/175; 604/523; 604/533

(57) ABSTRACT

Catheters and their uses for vascular access to a patient, especially during dialysis procedures, are described. The catheter contains a continuous, multi-geometric flow path profile along the axis of the catheter. Thus, the shape and material forming the fluid pathway of the catheter can change anywhere along the axis of the catheter. With multi lumen catheters, this profile transforms from a substantially D-shaped cross section in the lumen of the catheter shaft to a substantially circular-shaped cross section in the extension leg. The materials used in the catheter can also transform from an opaque material used in the catheter shaft to a clear or transparent material that is used in the extension leg. The transition between these shapes and materials can be seamless, eliminating the need for a mechanical joint that is needed because the catheter shaft and catheter extension leg(s) are comprised of a single component. Other embodiments are described.

### CROSS-REFERENCE TO RELATED APPLICATION

This patent application claims priority of U.S. Provisional Application Ser. No. 60/976,290, filed Sep. 28, 2007, the entire disclosure of which is hereby incorporated by reference.

### FIELD

This application generally relates to medical devices and methods for making and using such medical devices. More particularly, this application relates to medical device used for vascular access, commonly known as a catheter, and methods for making and using such medical devices.

### BACKGROUND

Catheters are hollow, flexible tubes for insertion into a body cavity, duct, or vascular system to form a fluid passageway. A catheter contains three main components including the (1) catheter shaft, (2) the hub or bifurcation, and (3) extension leg(s). The catheter shaft is that portion of the catheter which is inserted into the vascular system and provides subcutaneous access. The catheter shaft can contain a single lumen forming a single flow path or can contain multiple lumens forming several flow paths. The shaft can comprise several types of materials such as silicone or polyurethane. Regardless of the material, however, the shaft typically contains a significant percentage of barium sulfate or other radio-opaque substance that helps define the location of medical device in the vascular system when it is subjected to fluoroscopy or x-rays.

The extension leg(s) of the catheter remains outside of the body of the patient and is connected to the catheter shaft by means of a catheter hub. This connection is typically formed with an over molding process in which the hub forms a thermal bonded junction or a chemical bond between the components. The extension leg(s) are typically formed from a transparent material, such as silicone or polyurethane, which allows the user to visualize the fluid flow through the catheter.

Another typical feature of catheters is the difference in the cross-sectional profile of the flow path between the components. The extension leg(s) typically maintain a circular cross section while the catheter shaft (at least in the case of a two lumen configuration) is produced with each lumen having a D-shaped cross section. The result of this configuration essentially divides the catheter into two separate and equal flow paths. The catheter hub forms the transition between these two cross-sectional geometries (i.e., from a D-shape to a circular shape).

### SUMMARY

The application describes catheters and their uses for vascular access to a patient, especially during dialysis procedures. The catheter contains a continuous, multi-geometric flow path profile along the axis of the catheter. Thus, the shape and material forming the fluid pathway of the catheter can change anywhere along the axis of the catheter. With multi lumen catheters, this profile transforms from a substantially D-shaped cross section in the lumen of the catheter shaft to a substantially circular-shaped cross section in the extension leg. The materials used in the catheter can also transform from an opaque material used in the catheter shaft to a clear or transparent material that is used in the extension leg. The transition between these shapes and materials can be seamless, eliminating the need for a mechanical joint that is needed because the catheter shaft and catheter extension leg(s) are comprised of a single component.

### BRIEF DESCRIPTION OF THE DRAWINGS

The following description can be understood in light of FIGS. 1-11, in which:

FIG. 1 illustrates some embodiments of a multi-geometric profile catheter containing the transition of one material to another;

FIG. 2 illustrates some embodiments of a multi-geometric profile catheter containing the transition of one material to another while maintaining the same cross section;

FIG. 3 illustrates some embodiments of a multi-geometric profile catheter with a double-lumen;

FIG. 4 illustrates an isometric view, top view, and sectional view of some embodiments of a multi-geometric profile catheter;

FIG. 5 illustrates some embodiments of a tunneler with a reverse tunneling design;

FIG. 6 illustrates an isometric assembly view of some embodiments of a multi-geometric profile catheter when combined with a tunneler;

FIG. 7 depicts a front and side view of some embodiments of a wing hub;

FIG. 8 shows an isometric view of some embodiments of a luer hub;

FIG. 9 illustrates some embodiments of a single-lumen, multi-geometric profile catheter;

FIG. 10 contains an isometric view of the assembled single-lumen, multi-geometric profile catheter of FIG. 9; and

FIG. 11 contains an isometric view of some embodiments of a low profile tube clamp.

Together with the following description, the Figures may demonstrate and explain the principles of the multi-geometric profile catheters. In the Figures, the thickness and configuration of components may be exaggerated for clarity. The same reference numerals in different drawings represent the same element, and thus their descriptions will not be repeated.

### DETAILED DESCRIPTION

The following description supplies specific details in order to provide a thorough understanding. Nevertheless, the skilled artisan would understand that the medical devices and methods for making and using such devices can be implemented and used without employing these specific details. Indeed, the technology can be practiced by modifying the illustrated method and resulting product and can be used in conjunction with apparatus and techniques conventionally used in the industry. For example, the description focuses on geometries containing a transition from a D-shape to a circular shape cross sectional configuration, and from an opaque to a clear material. But it could easily be adapted for any number or type of cross sectional geometries and any number or type of materials.

The multi-geometric profile catheters contain a first section with a first cross-sectional shape and a second section with a second cross-sectional shape that can be different than the first shape. Between the first and second section is a first transition section where the cross-sectional shape changes from the first to the second cross-sectional shape. The first transition section can have a single cross-sectional shape or can have multiple cross-sectional shapes.

The multi-geometric profile catheters also contain a third section comprising a first material and a fourth section comprising a second material that can be different than the first material. Between the third and fourth sections is a second transition section where the material changes from the third material to the fourth material. The second transition section can have a single material or can have multiple materials.

The first shape of the multi-geometric profile catheters can be any cross-sectional shape known in the art. In some embodiments, the first cross-sectional shape can be a substantially circular oval, or a D-shape. In other embodiments, such as where the catheter contains multiple lumens, the first cross-sectional shape comprises a substantial D-shape.

The second shape of the multi-geometric profile catheters can be any cross-sectional shape known in the art. In some embodiments, the second cross-sectional shape can be a substantially D-shape, oval, or a circular shape. In other embodiments, such as where the catheter contains multiple lumens, the second cross-sectional shape comprises a substantially circular shape.

The first transition section of the multi-geometric profile catheters can have any single or multiple cross-sectional shape known in the art. In some embodiments, the first transition section can contain a substantially oblong, oval, circular, or a D-shape. In other embodiments, such as where the catheter contains multiple lumens, the first transition section contains an oblong shape or any intermittent geometry that lofts from one cross sectional geometry to another.

The first transition section can be positioned at any location of the catheter that facilitates the desired form, fit, or function of the catheter. In some embodiments, the first transition section is located between the catheter shaft and the extension legs. This transition section can range from about one to about six inches in length. In other embodiments, the transition section can be located along the axis of the catheter shaft, such as a location where the catheter requires a more or less ridged cross sectional geometry to address repeated flexing or contact with delicate tissue, respectively.

The first material of the multi-geometric profile catheters can be any opaque or radio opaque material known in the art. In some embodiments, the first material can be silicone, polypropylene, polyethylene, polyurethane, or combinations thereof. In other embodiments, the first material comprises any opaque Carbothane® material.

The second material of the multi-geometric profile catheters can be any clear, translucent or transparent material known in the art. In some embodiments, the second material can be silicone, polypropylene, polyethylene, polyurethane, or combinations thereof. In other embodiments, the first material comprises any clear Carbothane® material.

The second transition section of the multi-geometric profile catheters can have any combination of materials known in the art. In some embodiments, the second transition section can contain any known opaque material or any known transparent material. In other embodiments, the second transition second contains an opaque material in a first portion (proximate the first material) and a transparent material in a second portion (proximate the second material).

The second transition section can be positioned at any location of the catheter that facilitates the desired form, fit, or function of the catheter. This transition section can range from about one to about six inches in length. In other embodiments, the transition section can be located along the axis of the catheter shaft, such as any location where the catheter requires a more or less ridged cross sectional geometry to address repeated flexing or contact with delicate tissue, respectively. The transition between the different shapes (the first transition section) and the different materials (the second transition section) can be at the same location or different location. In some embodiments, the first and second transition sections are located in substantially the same location in the catheter. In other embodiments, the first and second transition sections can be located at different locations that are separated according to the desired form, fit, or function of the catheter.

In some embodiments, the size of the catheter can change along the axis of the catheter. The size can change from one section to another or within any given section. The size can also increase or decrease along the entire length or along discrete portions of the catheter. As one example, the size of the catheter could be a first size in the first (or third) section and a second size in the third (or fourth) section and then increase or decrease from the first to the second size in the first (or second) transition section.

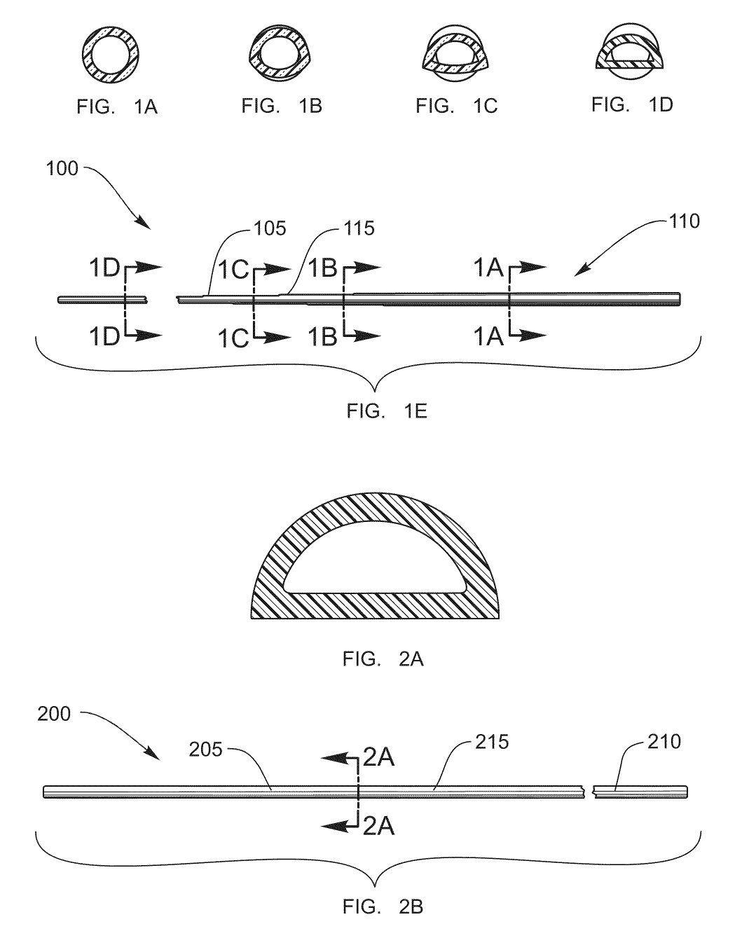

Some embodiments of the multi-geometric profile catheters are illustrated in FIG. 1. FIG. 1 depicts a catheter 100 which contains a first section 105 that has a D-shaped cross-sectional geometry and is made of an opaque material. The exemplary geometry of the first section 105 is shown in the cross-section D-D. The catheter 100 also contains a second section 110 that has a substantially circular shape cross-sectional geometry and is made of clear material. The exemplary geometry of the second section 110 is shown in the cross-section A-A.

The catheter 100 also contains a transition section 115 where the cross sectional shape and the material change from the first section 105 to the second section 110. The exemplary geometry of the transition section 115 is shown in the cross-sections C-C and B-B. As depicted in FIG. 1, the geometry of the transition section 115 can vary along its length from the first section 105 to the second section 110. This transition section eliminates the need for a bonded, insert molded, or mechanical junction member that is often used in conventional catheters.

In the embodiments illustrated in FIG. 2, the extrusion of the catheter 200 contains a single shape (a low profile D-shape as shown in A-A) and just transforms from the first material in the first section 205 to the second material in the second section 210. The transition section 215 changes gradually from the opaque material in the first section to the clear material of the second section 210. Again, the transition section 215 helps to eliminate the need for a bonded, insert molded, or mechanical junction member, as well as allowing for a low profile tube clamp (as described herein) to be used with the D-shaped profile of the catheter.

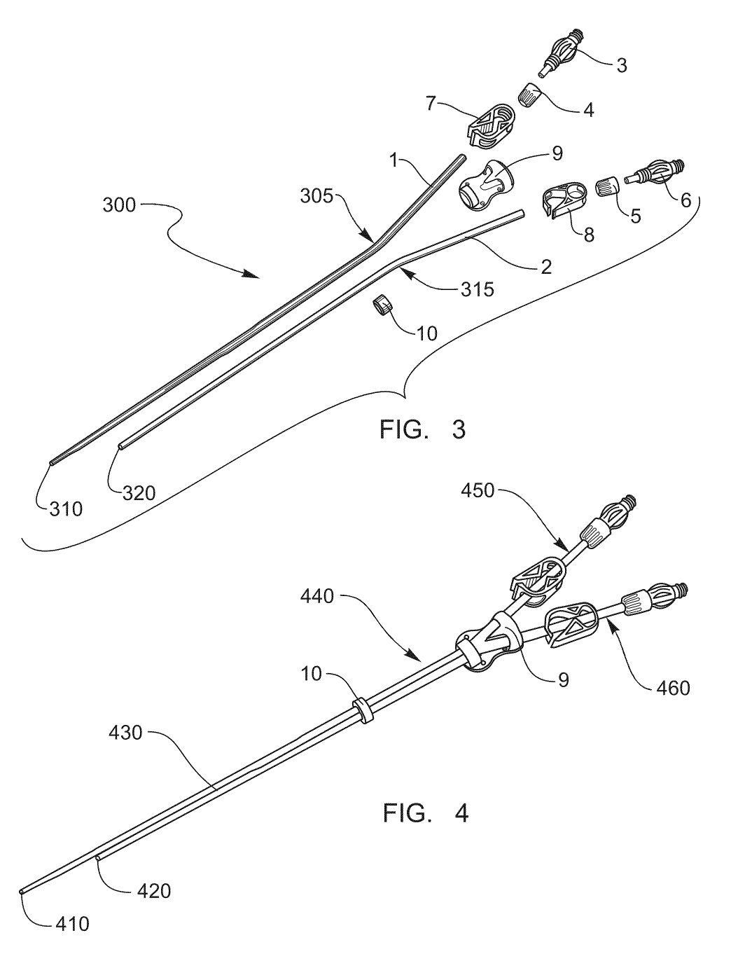

Other embodiments of the multi-geometric profile catheters are depicted in FIGS. 3 and 4. In these embodiments, the components of a reverse tunneling, two lumen dialysis catheter 300 are illustrated in FIG. 3 in an unassembled configuration and in an assembled configuration in FIG. 4. The catheter 300 contains a venous member 1 that transitions from a D-shaped catheter shaft to round-shaped extension leg. The venous member 1 can be extruded (or otherwise manufactured) as a single member. The catheter 300 also contains a venous dialysis tip 310 at the distal end of the venous member 1. As shown in FIG. 3, the catheter 300 contains a transition section 305 for the venous member 1 that transition both from an opaque material to a clear material and from a D-shaped cross sectional geometry to a circular shaped cross sectional geometry.

The catheter 300 also contains an arterial member 2 that transitions from D-shaped cather shaft to a round-shaped catheter extension leg. The arterial member 2 can be extruded (or otherwise manufactured) as a single member. The catheter 300 also contains an arterial dialysis tip 320 at the distal end. As shown in FIG. 3, the arterial member 2 contains a transition section 315 that transitions both from an opaque material to a clear material and from a D-shaped cross sectional geometry to a circular shaped cross sectional geometry.

The venous member 1 and arterial member 2 can be joined together by any known method. In some embodiments, these two components are joined by using any known chemical and/or thermal bonding that can be performed as part of the manufacturing process. In other embodiments, the venous member 1 and arterial member 2 can be extruded together in the same extrusion process.

The catheter 300 can be manufactured with several configurations. One of these configurations include joining these two components (venous member 1 and arterial member 2) in a staggered configuration so that the venous/arterial legs are joined with an offset between the distal venous tip 410 and the distal arterial tip 420, as shown in FIG. 4. In this configuration, the catheter shafts (collectively, 440) can be bonded together with thermally or with a solvent up to the desired location 430. Another configuration includes a split tip configuration where the proximate ends of the venous/arterial legs are joined together with an offset. The extension legs 450 and 460 remain separated and can be trimmed to the desired length as known in the art.

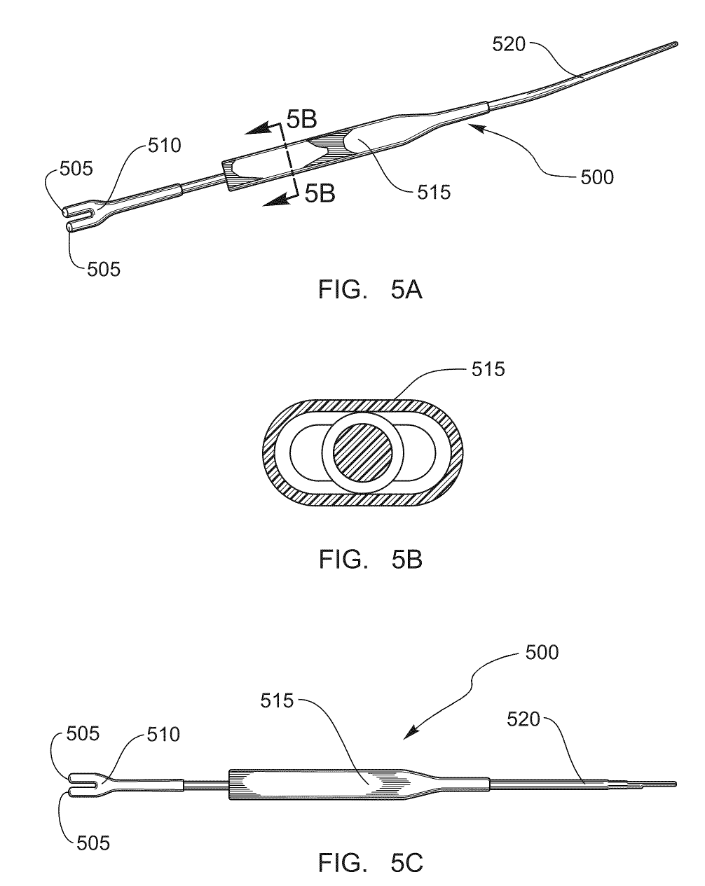

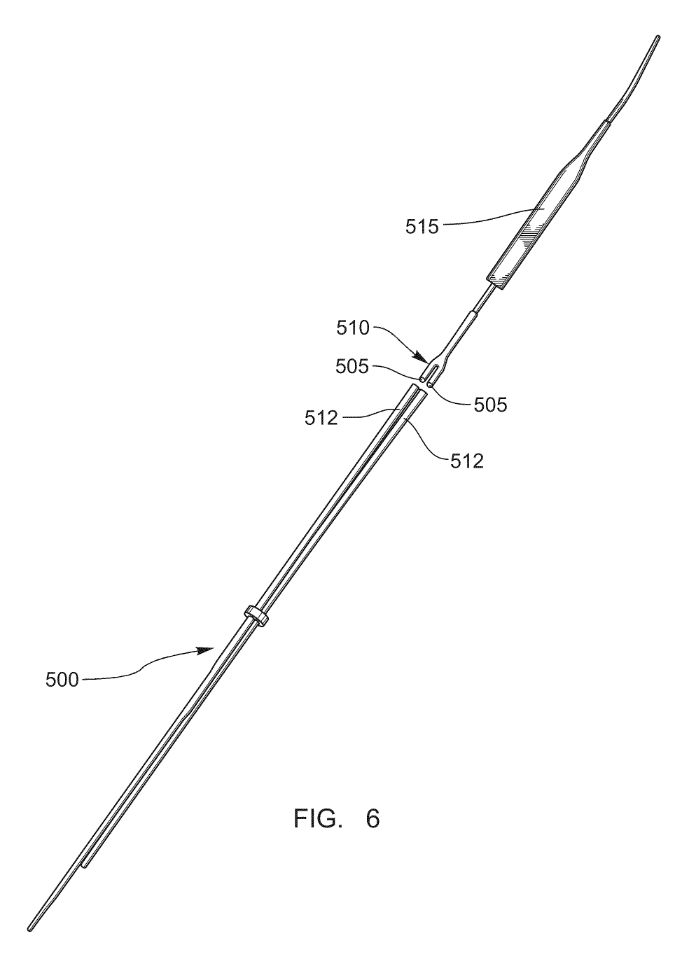

The catheters described herein can be inserted in the body of a patient using any known tunneler that is compatible with the catheter. One example of such a tunneler (for the catheters in FIGS. 3-4) is depicted in FIG. 5 where the tunneler 500 contains two protrusions 505 that are attached to the tunneler member 520 in any known manner, including by means of insert molding or bonding. As shown in FIG. 6, these protrusions 505 are inserted into the untrimmed extension leg(s) 512 of a catheter 600 prior to the tunneling procedure. The extension legs 512 can be inserted to any length up to the extension leg connector 510. The sheath 515 shown in FIG. 5 can be configured to be oval shaped so that it is capable of retracting over the both ends of the extension leg(s) 512, thereby reducing friction while pulling the extension leg(s) through the tunnel. Once the extension leg(s) 512 are through the tunnel, the tunneler 500 can be removed and the extensions leg(s) 512 trimmed to the desired length. Upon trimming the extension leg(s) 512 of the catheter 600 to the desired length, the proximal ends of the extension legs of the catheter are then locked into place. This locking procedure comprises inserting the extension leg(s) 512 through the bifurcation/suture wing 9 shown in FIG. 3. The final location of the bifurcation/suture wing 9 (in a locked position) is shown in FIG. 4.

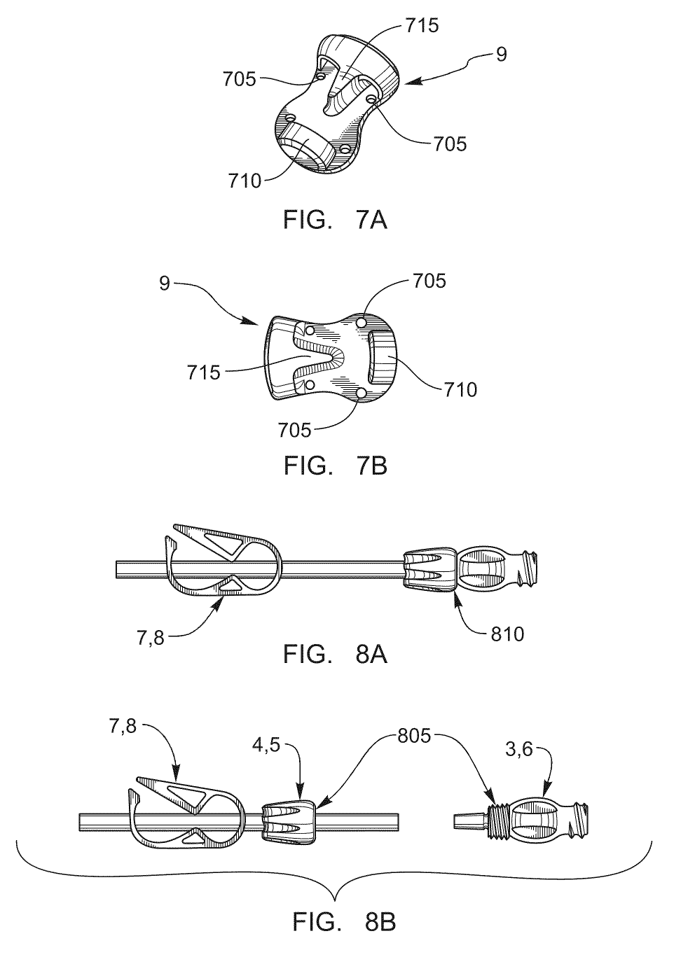

The details of the bifurcation/suture wing 9 shown in FIGS. 3-4 are also depicted in several close-up views in FIG. 7. The bifurcation/suture wing 9 contains suture holes 705 with any desired size and width that allow the catheter to be secured to the patient. The bifurcation/suture wing 9 can also contains other known components, including a retainer 710 for retaining the catheter shafts and a separator 715 for the extension legs.

Once the bifurcation/suture wing 9 is placed in the desired location, the tube clamps and hub connector locks are then placed on the extension legs. After inserting the extension legs through the bifurcation/suture wing 9, a venous tube clamp 7 and an arterial tube camp 8 (both are shown in FIGS. 3-4 with a detailed view of these tube clamps depicted in FIG. 11) are placed over the trimmed extension leg(s) as known in the art. As depicted in FIG. 11, the tube clamps 7 and 8 can have a lower profile than other tube clamps.

A venous luer hub connector lock 4 and an arterial luer hub connector lock 5 can then be connected to the respective end portions of the extension legs as known in the art. The venous luer hub connector 3 and an arterial luer hub connector 6 are then placed onto the respective ends of the extension leg(s) to form a hermetic seal. The venous luer hub connector 3 (and/or arterial luer hub connector 6) can be secured into place in any known manner, including by means of a mechanical lock 805 that is formed between the luer hub connector lock 3 (or 4) and the luer hub connector 6 (or 5) to obtain the assembled structure 810 depicted in FIG. 8.

Finally, a tissue cuff 10 can be assembled during the manufacturing process by means of thermal or chemical bonding and attached to the catheter. The tissue cuff promotes tissue ingrowth, secures the catheter in place, as well as in some instances, helps provide protection against infections related to vascular access catheters. Optionally, additional locking features could be used in the catheters. Examples of these locking features include snap fits or detents that could be implemented to reduce the ability for the luer hub connectors from separating from the extension leg(s).

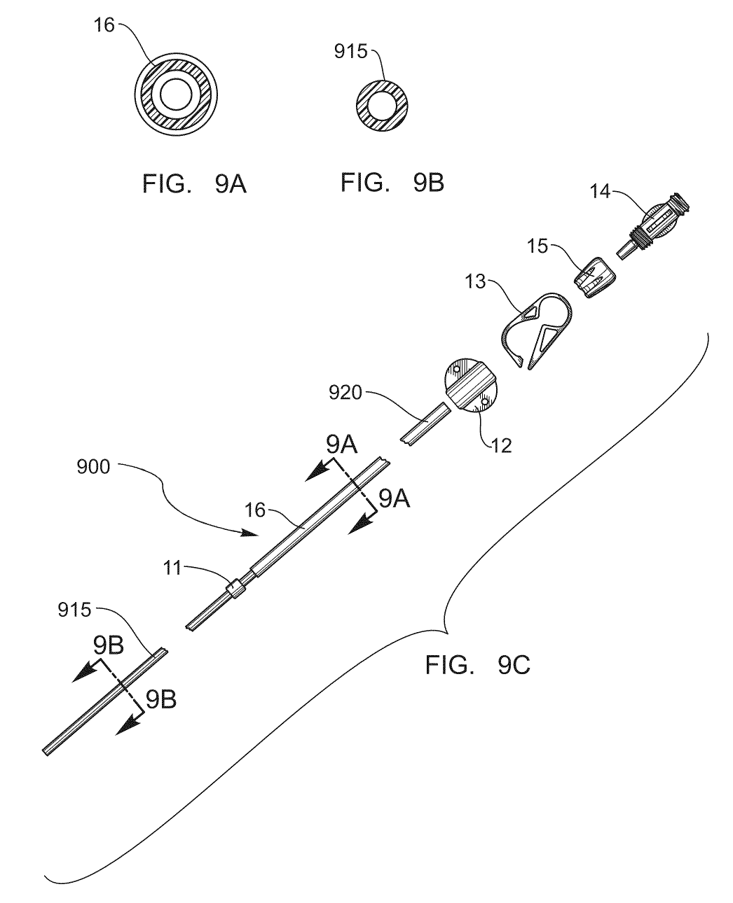

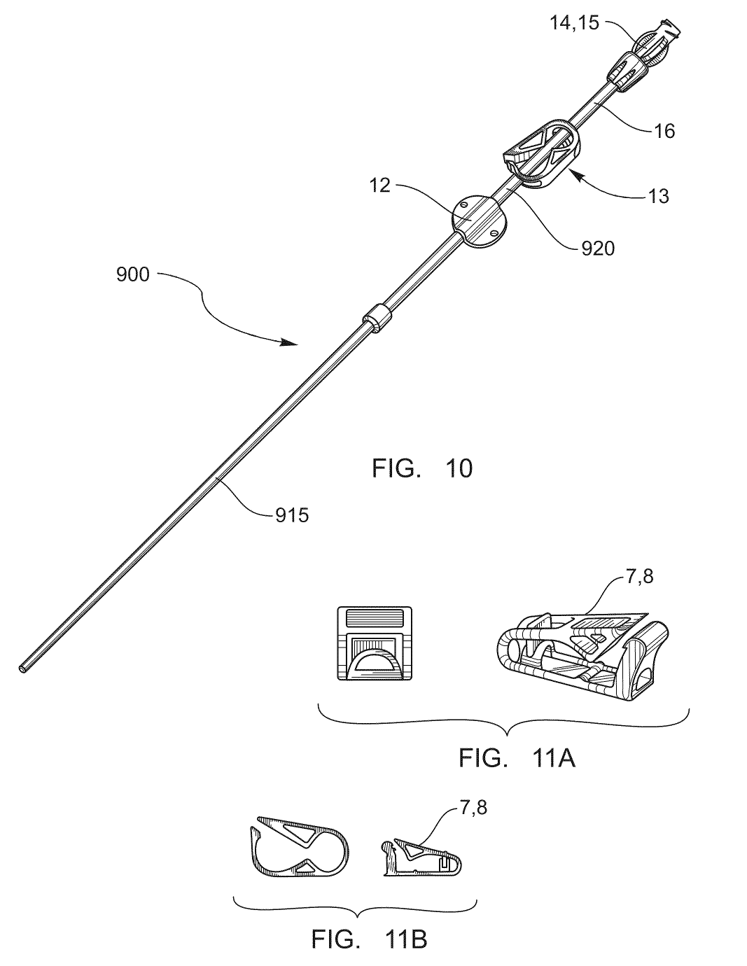

Other embodiments of the multi-geometric profile catheters are depicted in FIGS. 9-10. These Figures depict a single-lumen catheter 900 with a transition in the size and the material of manufacture along the axis. The catheter shaft and extension leg is extruded (or otherwise manufactured) as a single member 16 transitioning from a first diameter (with a first cross sectional shape) in a first section 915 of the catheter to second, larger diameter (with a second cross sectional shape) in a second section 920 of the catheter 900. The catheter 900 also contains a transition section 905 between the first and second sections, whether the diameter(s) of the transition section 905 is between the first diameter and the second diameter. The transition section 905 also changes from an opaque material that is used in the first section 915 of the catheter 900 to a clear material that is used in the second section 920. In some embodiments, the shape of the catheter could change along the axis similar to the other catheters described herein.

In these embodiments of a single-lumen catheter, the tunneler depicted in FIG. 5 could be modified to contain only a single protrusion and be used with catheter 900. This single protrusion could be attached to the tunneler by any suitable means, including insert molding or bonding. This protrusion could then be inserted into the untrimmed extension leg of catheter 900 prior to tunneling. The sheath of the tunneler could then be retracted over the end of the extension leg, which reduces friction while pulling the extension leg through the tunnel. Once the extension leg is through the tunnel, the tunneler can be removed and the extensions leg trimmed to the desired length.

Upon trimming the extension leg to its final length, the proximal end of the extension leg of the single member 16 is locked into place. That locking process includes inserting the extension leg through suture wing 12 as shown in FIG. 9. The suture wing 12 also contains suture holes of a standard size and width that allow the catheter assembly to be secured to the patient. A tube clamp 13 (substantially similar to the tube clamps detailed in FIG. 11) is then placed over the trimmed extension leg, followed by a luer hub connector lock 15. A luer hub connector 14 is then inserted into the end of the extension leg, forming a hermetic seal. The luer hub connector 14 can be secured into place by means of a mechanical lock formed by assembling the luer hub connector lock 15 onto the luer hub connector 14. The additional locking features described above and a tissue cuff 11 could also be incorporated into the catheter 900. FIG. 10 shows the single lumen catheter 900 as a finished assembly with the extension leg trimmed and the catheter in a final configuration after being placed in the body of a patient.

The catheters described herein provide a much safer catheter in terms of fluid leak or air embolism because the catheter shaft and extension leg are formed from a single member and the tube clamp is assembled below the joint formed by luer hub 4. This configuration provides a secondary seal if the luer hub connection were to become cracked or otherwise damaged while the catheter was not being used.

Because of this single member, the catheters do not experience some of the failures and problems that occur with conventional three-component catheters. The catheter shaft, joint hub, and extension tube(s) in these catheters are three separate components because after the reverse tunneling and trimming procedure is complete, the user must assemble the three components together to form a single catheter. The shaft and extension legs are joined together by means of a mechanical joint provided by the joint hub. Because this is a mechanical joint, the potential risk of joint detachment exists—especially in the case of a multi lumen configuration—which can result in blood loss or an air embolism.

This joint hub component itself can also cause problems. The connection of this joint hub adds an additional step to the catheter insertion/connection procedure (increasing surgical time and expense), requires an additional connection which may leak or separate from the catheter tube due to external loads on the hub (such as by pulling or snagging), and is a relatively complex part, which increases the cost of the catheter since it is difficult to manufacture. As well, the joint hubs can contribute to safety concerns. Tubing clamps are often placed on the extension leg(s) of a catheter as an added safety feature since they can prevent blood loss if the proximal end of an extension leg was to become damaged. But there is no ability to place a tubing clamp on the distal location of the joint hub between the catheter shaft and the extension legs.

Another failure with multi lumen catheters are cross-over leaks (molding defects in the wall separating the lumens), which can result in unintended fluid communication between the lumens. Another failure occurs during the procedure for placing catheters into the vascular system, especially for dialysis. See, for example, U.S. Pat. No. 6,872,198. For optimal performance in dialysis, the catheter tips of either a double lumen catheter or two single lumen catheters should be placed in close proximity to the heart. While double lumen catheters allow for a single insertion into the desired vein, double lumen catheters often do not permit optimal catheter tip placement because the optimal top position varies from patient to patient. And non-optimal tip position significantly lowers flow values and results in less effective dialysis treatment. With two independent single lumen catheters, the problem of tip placement is diminished. But this method requires two separate venous insertions, two tunnels, and two of each instrument needed for the surgical procedure. This increases the surgical time, creates two entry sites (which doubles the risk of post-surgical infection), and the two catheters together are significantly larger in diameter than a single double-lumen catheter.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, examples are meant to be illustrative only and should not be construed to be limiting in any manner.

1. A catheter, comprising:
a shaft comprising a lumen have a substantially D-shaped cross section;
an extension leg having a substantially circular-circular shaped cross section; and
a transition section between the shaft and the extension leg that does not contain a joint hub.

2. The catheter of claim 1, wherein the transition section transitions from a substantially D-shaped cross section near the shaft to a substantially circular-circular shaped cross section near the extension leg.

3. The catheter of claim 1, further comprising another lumen and another extension leg.

4. The catheter of claim 1, wherein the shaft, transition section, and extension leg are made from a single extruded member.

5. The catheter of claim 1, wherein the shaft and the extension leg are comprised of different materials.

6. A catheter, comprising:
a shaft comprising a lumen with a first cross section having a first shape;
an extension leg having a second cross section shaped differently than the first cross-section; and
a transition section between the shaft and the extension leg that comprises a substantially continuous, multi-geometric cross sectional flow path.

7. The catheter of claim 6, wherein the transition section changes both in shape and material between the shaft and extension leg.

8. The catheter of claim 6, wherein the first cross section has a substantially D-shape and the second cross-section has a substantially circular cross section.

9. The catheter of claim 6, further comprising another lumen and another extension leg.

10. The catheter of claim 6, wherein the shaft, transition section, and extension leg are made from a single extruded member.

11. The catheter of claim 6, wherein the first cross section has substantially circular shape with a first diameter and the second cross-section has a substantially circular shape with a different diameter.

12. The catheter of claim 6, further comprising a luer hub that can be attached to the extension leg.

13. A medical device, comprising:
a catheter containing a shaft comprising a lumen with a first cross section having a first shape, an extension leg having a second cross section shaped differently than the first cross-section, and a transition section between the shaft and the extension leg that comprises a substantially continuous, multi-geometric cross sectional flow path; and
a tunneler having a distal tapered end and a proximal end with a base containing a gripping surface.

14. The device of claim 13, wherein the transition section changes both in shape and material between the catheter shaft and extension leg.

15. The device of claim 13, further comprising another lumen and another extension leg.

16. The device of claim 15, wherein the tunneler contains multiple flexible shaft members extending from the base for insertion into the catheter extension legs.

17. The device of claim 13, wherein the shaft, transition section, and extension leg are made from a single extruded member.

18. A method, comprising:
providing a catheter containing a shaft with multiple lumens having a first cross section, multiple extension legs having a second cross section shaped differently than the first cross-section, and a transition section between the shaft and the extension legs that comprises a substantially continuous, multi-geometric cross sectional flow path;
providing a tunneler containing multiple flexible shaft members; and
inserting the tunneler shaft members into the extension legs.

19. The method of claim 17, further comprising:
pulling the tunneler through a pair of incisions in the body;
detaching the tunneler from the catheter extension legs;
trimming the catheter extension legs to a desired length;
attaching a suture wing hub over the extension legs;
attaching a clamp over the extension legs; and
attaching a lockable luer hub onto the distal end of the extension legs.

20. The method of claim 18, further comprising:
pulling the tunneler through a pair of incisions in the body;
detaching the tunneler from the catheter extension legs;
trimming the catheter extension legs to a desired length;
attaching a suture wing hub over the extension legs;
attaching a clamp over the extension legs; and
attaching a lockable luer hub onto the distal end of the extension legs.

21. A catheter, comprising:
a shaft comprising a lumen have a cross section with a first diameter;
an extension leg having a cross section with a second diameter that is different than the first diameter; and
a transition section between the shaft and the extension leg that does not contain a joint hub.

22. The catheter of claim 21, wherein the second diameter is larger than the first diameter.

23. The catheter of claim 21, wherein the catheter shaft, transition section, and extension leg are made as a single member.

24. The catheter of claim 21, wherein the shaft and the extension leg are comprised of different materials.
